(19) United States
(12) Patent Application Publication
(10) Pub. No.: US 2007/0129693 A1
(43) Pub. Date: Jun. 7, 2007

(54) Controlled needle-free eye injector

(76) Inventors: Ian W. Hunter, Lincoln, MA (US); Andrew T. Taberner, Lexington, MA (US); Dawn M. Wendell, Farmington, CT (US); Nora Catherine Hogan, Boston, MA (US); Brian D. Hemond, Lexington, MA (US); Ching-Hua Tseng, Belmont, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/598,556

(22) Filed: Nov. 13, 2006

Related U.S. Application Data
(60) Provisional application No. 60/735,713, filed on Nov. 11, 2005.

(51) Int. Cl. A61M 35/00

(52) U.S. Cl. 604/294; 604/181

(57) ABSTRACT

An eye injector for transferring a substance across the surface of the eye of an animal includes a needle-free injector and a patient positioning mechanism. The needle-free injector includes a reservoir for storing the substance to be transferred, a nozzle in fluid communication with the reservoir; and a controllable electromagnetic actuator in communication with the reservoir. The eye injector can also include a targeting light source for directing a beam of light into a pupil of the eye. The needle-free injector can be positioned adjacent to the lens of the eye, therefore producing a jet that intersects with the beam of light at a point in the retina.

### RELATED APPLICATION

This application claims the benefit of U.S. Provisional Application No. 60/735,713, filed on Nov. 11, 2005. The entire teachings of the above application are incorporated herein by reference.

### BACKGROUND OF THE INVENTION

Ocular injections are often used for treatment of various diseases of the eye, for example glaucoma and various diseases causing vision loss. The treatments of such diseases generally require the injection of a treatment drug into the eye, using a conventional needle. Delivery of the drug by a needle causes increased risk of infection to the eye, as well as further damage to different regions of the eye. Further, injections with a needle cause increased anxiety and potentially pain to a patient.

### SUMMARY OF THE INVENTION

A needle-free jet injector may be used in order to reduce the risk of infection or damage to a patient's eye, as well as reducing the pain and anxiety of a needle into the eye. A delivery system for transferring a substance into an eye of an animal includes a needle-free injector. The needle-free injector includes a reservoir for storing the substance to be transferred to the eye, a nozzle in fluid communication with the reservoir, and a controllable electromagnetic actuator in communication with the reservoir. The delivery system can further include patient positioning mechanism to position the needle-free injector against a head of patient to direct the needle-free injector to inject a jet of the substance into the eye.

The delivery system can further include a targeting light source that directs a beam of light through the pupil of the eye. The light source, may for example, be a laser such an as infrared laser beam. The needle-free injector is positioned adjacent to a lens of the eye. A jet produced by the needle-free injector and a beam of light produced by the targeting light source intersect at a point at or near the retina of the eye. A jet of the substance is then injected through the vitreous humor of the eye to the retina of the eye.

The depth of injection is controllable. The injection is most often made to a depth of about 30 millimeters in the eye, since that is the approximate depth of most eyes. The injected substance can be a drug or a powder substance.

The electromagnetic actuator includes a stationary magnet that provides a magnetic field. The electromagnetic actuator further includes a coil assembly, that is slidably disposed with respect to the magnet assembly. The coil assembly receives an electrical input and generates in response a force corresponding to the received input. The force results from interaction of an electrical current within the coil assembly and the magnetic field and causes a needle-free transfer of the substance between the reservoir and the eye. The electromagnetic actuator forces the substance through a nozzle producing a jet having sufficient velocity to pierce the surface of the eye. The delivery of a substance may be made between 30-50 milliseconds. The rise time of the generated force may be less than about 5 milliseconds. The force is of sufficient magnitude and duration to transfer a volume of up to at least about 300 micro liters of the substance to the eye.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows an illustration of one embodiment of an eye injector;

FIG. 2 is a schematic block diagram of one embodiment of a needle-free injector that is part of an eye injector;

FIGS. 3A and 3B are cross-sectional diagrams of one embodiment of a controllable electromagnetic actuator usable with the eye injector of FIG. 1;

FIG. 4A is a graph depicting a current-versus-time profile of an exemplary electrical input to the controllable electromagnetic actuator of FIG. 3A;

FIG. 4B is a graph depicting a pressure-versus-time profile of an exemplary pressure generated within a reservoir used in the transfer of a substance, the pressure being generated by the controllable electromagnetic actuator responsive to the electrical input of FIG. 4A;

FIG. 5 is a partial cut-away perspective diagram of an embodiment of a needle-free injector of the eye injector of FIG. 1;

FIG. 6 is a partial cut-away perspective diagram of an alternative embodiment of a needle-free injector that is part of the eye injector of FIG. 1;

FIG. 7 is a more detailed partial cut-away perspective diagram of the controllable electromagnetic actuator provided in the device of FIG. 6 coupled to a syringe;

FIG. 8 is a rear perspective diagram of an embodiment of the controllable;

FIG. 9 is a graph depicting a current-versus-time profile for a multi-shot eye injector;

FIG. 10 illustrates one embodiment of an eye injector being used to deliver a substance into the eye;

FIG. 11 shows a simulated injection being injected into a simulated eye; and

FIG. 12 shows a slit lamp that can be used in conjunction with an eye injector.

### DETAILED DESCRIPTION OF THE INVENTION

A description of preferred embodiments of the invention follows.

A needle-free eye injection device, or eye injector, is configured to inject a substance into the eye of an animal body. The eye injector, which uses an electromagnetic actuator allows for a controlled and noninvasive delivery of a substance into the eye. Injection devices include devices having one or more needles configured to pierce the eye prior to injection of the substance (e.g., typical hypodermic needle). Other injection devices are configured to inject a substance beneath the skin without first piercing the skin with a needle (i.e., needle-free). It should be noted that the term “needle-free” as used herein refers to devices that inject without first piercing the eye with a needle or lance. Some needle-free injection devices rely on a pioneer projectile ejected from the device to first pierce the eye. Other needle-free injection devices rely on pressure provided by the drug itself.

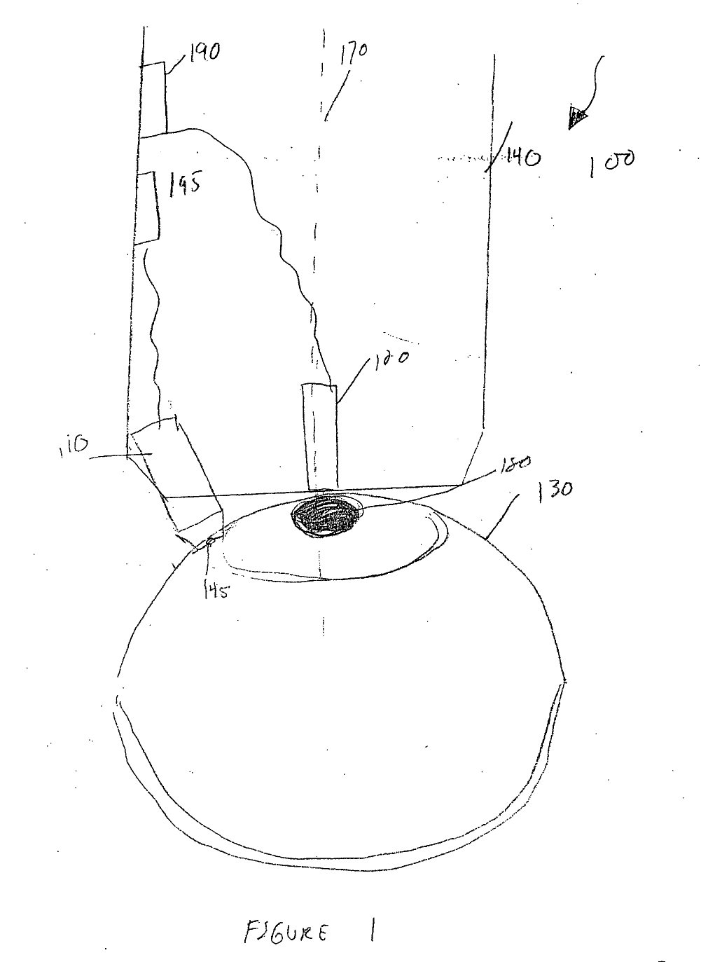

FIG. 1 shows an embodiment of a needle-free eye injection device, or eye injector 100. The eye injector 100 includes a housing 140. The housing 140 is shaped and sized to be used with an eye of an animal 130. The housing 140 can be any suitable shape or size that is compatible with the eye 130. The housing 140 can be shaped to be a handheld device, or may also contain components that allow it to fit within or to be used in conjunction with additional optical instruments, for example a slit lamp photometer.

The eye injector 100 also includes a needle free injector 110. The needle-free injector 1 10 is used to produce a jet with a strong enough velocity to pierce the surface of the eye 130 without a needle. A tip, or nozzle or the needle-free injector 110 can protrude from the housing 140 and touch the surface of the eye 130. Alternatively, the needle-free injector 110 can be completely within the housing 140, as long as there is an open path within the housing 140 that allows for a jet to be injected into the eye 130. The needle-free injector has at least one orifice 145 through which a substance is injected.

The needle-free injector 110 can be positioned at an angle within the housing 140, which results in it being adjacent to a lens 150 of the eye 130. Alternatively, the needle-free injector 110 can be positioned parallel to a major axis 170 of the eye 130. In this case, the orifice 145 might be placed in a lateral position on a nozzle or end of the needle-free injector 110, such that the injection is made at an angle.

The eye injector 100 also includes a targeting light source 120. The targeting light source 120 may be, for example, a laser beam such as an infared laser. The targetted light source can alternatively be any type of light source, that can safely target a beam of light into the eye without causing any damage or changes within the eye 130. The light source 120 targets a beam of light into a pupil 180 of the eye. The targeting light source 120 can be positioned along and parallel to the major axis of the eye 170. Alternatively, the targeting light source 120 need not be placed parallel to the major axis 170. It could be positioned at an angle on eye 130, as long as the beam of light produced by the targeting light source 120 intersects with the jet produced by the needle-free injector 110.

The targeting light source 120 can protrude from the housing 140 such that it touches the surface of the eye 130. Alternatively, the targeting light source can be positioned completely within the housing 140, such that it does not touch the eye. The housing 140 can contain one or more controllers 190 and 195 which allow, for control of one or both of the needle-free injector 110 and the targeting light source 120 by a user or the eye injector 100. For example, one controller may contain mechanisms to control both, such as switches to operate the needle-free injector 110 and the light source 120. Alternatively, both the needle-free injector 110 and the light source 120 can have their own respective controllers 195 and 190 that are connected to each.

The position of both the needle-free injector 110 and the targeting light source 120 may be adjustable within the housing 140. For example, the housing 140 may contain external levers or handles connect to both the needle-free injector 110 and the housing 140, that allow their respective positions to be adjusted with respect to the housing 140.

An outer diameter of the eye injector 100 can be about 30-40 millimeters and about 200 millimeters in length. The diameter and length of the eye injector 100, can however, be any suitable diameter or length to allow injection into the eye 130.

Alternatively or in addition, the housing 140 may not be needed. The needle-free injector 110 and the light source 120 can be coupled by other means. Further, they may be an accessory to an additional optical instrument, such as an opthamaloscope or a slit lamp photometer.

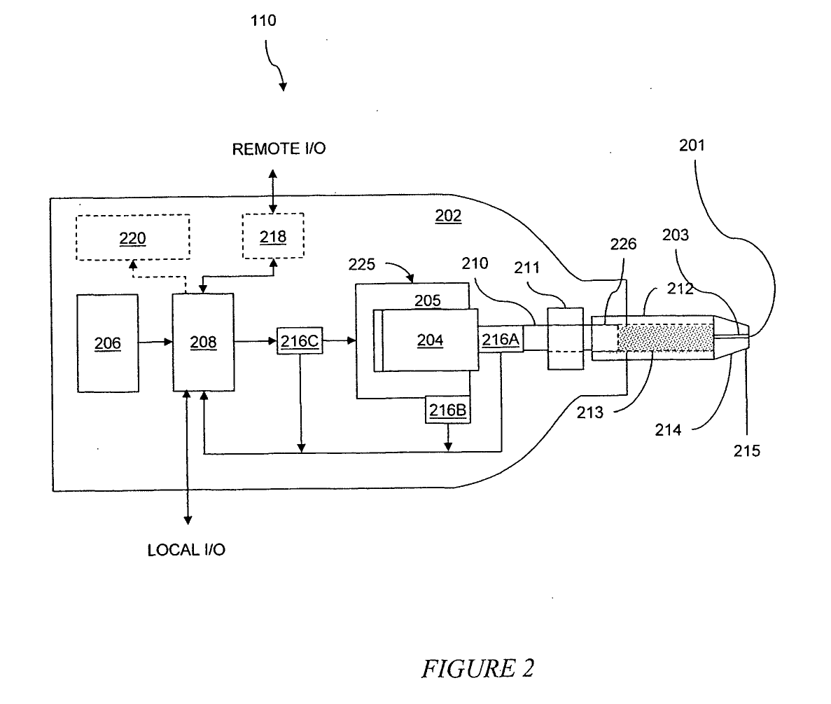

FIG. 2 shows a schematic block diagram of the needle-free injector 110 of the eye injector 100, used to transfer a substance across the surface of the eye 130. For example, the needle-free injector 110 can be used to inject a liquid formulation of an active principle, for example, a drug, into the eye 130 an animal. Alternatively or in addition, the same needle-free injector 110 can be used to collect a sample from the eye of the animal by withdrawing the collected sample through the surface of the eye 130 and into an external reservoir 213 that may be provided within the needle-free injector 110. If the needle-free injector 110 is positioned within the housing 140, as described in FIG.1, the needle-free injector can be removed from the housing 140 to obtain the sample from the reservoir 213. Examples of needle-free injectors that are used to inject and withdraw samples from a biological body are presented in U.S. application Ser. No. 11/352,916, filed Feb. 10, 2006 claiming the benefit of U.S. Provisional Application 60/652,483, filed Feb. 11, 2005 and U.S. application Ser. No. 11/351,887, also claiming the benefit of U.S. Provisional Application 60/652,483, all of which are herein incorporated by reference in their entirety.

The needle-free injector 110 typically includes a nozzle 214 to convey the substance through the surface of the eye 130 at the required speed and diameter to 25 penetrate the surface of the eye 130. Namely, substance ejected from the nozzle 214 forms a jet, the force of the jet determining the depth of penetration. The nozzle 214 generally contains a flat surface, such as the head 215 that can be placed against the eye and an orifice 201. It is the inner diameter of the orifice 201 that controls the diameter of the transferred stream. Additionally, the length of an aperture, or tube 203, defining the orifice 201 also controls the transfer (e.g., injection) pressure.

In some embodiments, a standard hypodermic needle is cut to a predetermined length and coupled to the head 215. One end of the needle is flush, or slightly recessed, with respect to the surface of the head 215 that contacts the skin to avoid puncturing the eye during use. The internal diameter of the needle (e.g., 100 μm) defines the diameter of the aperture, and the length of the needle (e.g., 5 mm) together with the aperture dimension controls the resulting injection pressure, for a given applicator pressure. In other embodiments, a hole can be drilled directly into the head 215 to reduce assembly steps. In general, the length of the orifice is selectable, for example ranging from 500 μm to 5 mm, while its diameter can range from 50 μm to 200 μm. In one particular embodiment, the diameter of the orifice is about 100 μm.

The smaller diameter of the orifice advantageously produces a smaller hole within the eye 130, thus reducing the chance of infection in the area of injection. By contrast, injectors that use needles to pierce the surface of the eye generally produce a hole of about 400 μm, resulting in higher chances of a larger infection to the injection site.

The nozzle 214 can be coupled to a syringe 212 defining a reservoir 213 for temporarily storing the transferred substance. The syringe 212 also includes a plunger or piston 226 having at least a distal end slidably disposed within the reservoir 213. Movement of the plunger 226 along the longitudinal axis of the syringe 212 in either direction creates a corresponding pressure within the reservoir 213. In some embodiments, the syringe 212 is integral to the needle-free injector 110. In other embodiments, the syringe 212 is separately attachable to the needle-free injector 110. For example, a commercially-available needle-free syringe 212 can be attached to the needle-free injector, such as a model reference no. 100100 syringe 212 available from Equidine Systems Inc. of San Diego, Calif.

The nozzle 214 can be releasably coupled to the syringe 212 or the distal end of the needle-free injector 110, such that different nozzles can be used for injecting and sampling (i.e., sucking), each different nozzle tailored for its intended use. Thus, a sampling nozzle may include a larger orifice 201, tapering into the lumen 203 thereby promoting a more efficient collection, or greater capacity sample.

Beneficially, a pressure is selectively applied to the chamber 213 using a controllable actuator. A specially-designed electromagnetic actuator 225 is configured to generate a high-pressure pulse having a rapid rise time (e.g., less than 1 millisecond). The actuator 225 can be used in needle-free injection devices that rely on high-pressure actuators to inject a formulation into the eye 130. Beneficially, the actuator is dynamically controllable, allowing for adjustments to the pressure-versus-time during actuation. At least one advantage of the electromagnetic actuator over other needle-free devices is its relatively quiet operation. Actuation involves movement of a freely suspended coil within a gap, rather than the sudden release of a spring or the discharge of a gas. Actuation of the freely-moving coil in the manner described herein results in quiet operation, which is an important feature as it contributes to reducing pain and anxiety during administration to the recipient and to others that may be nearby.

In more detail, the electromagnetic actuator 225 is configured to provide a linear force applied to the plunger 226 to achieve the transfer of the substance. Transfer of the force can be accomplished with a force-transfer member 210, such as a rigid rod slidably coupled through a bearing 211. The rod may be secured at either end such that movement of the actuator in either direction also moves the plunger 226. The bearing restricts radial movement of the rod 210, while allowing axial movement.

In some embodiments, the actuator 225 includes a stationary component, such as a magnet assembly 205, and a moveable component, such as coil assembly 204. A force produced within the coil assembly 204 can be applied to the plunger 226 either directly or indirectly through the rod 210 to achieve transfer of the substance. Generally, the actuator 225, bearing 211 and syringe 212 are coupled to a frame or housing 202 of the needle-free injector 110 that provides support and maintains fixed position of these elements during an actuation.

In some embodiments, the needle-free injector 110 includes a user interface 220 that provides a status of the device. The user interface may provide a simple indication that the device is ready for an actuation. For example, a light emitting diode (LED) coupled to a controller 208 can be enabled when sufficient conditions are satisfied for an injection. More elaborate user interfaces 220 can be included to provide more detailed information, including a liquid crystal display (LCD), cathode ray tube (CRD), charge-coupled device (CCD), or any other suitable technology capable of conveying detailed information between a user and the needle-free injector 110. Thus, user interface 220 may also contain provisions, such as a touch screen to enable an operator to provide inputs as user selections for one or more parameters. For example, one might measure and record the intraocular pressure of the eye. A user may identify various parameters related to dose, sample, or parameters of the eye. The user interface 220 can alternatively be positioned on the housing 140 as described in FIG. 1, if the needle-free injector 110 is positioned within the housing 140.

A power source 206 provides an electrical input to the coil assembly 204 of the actuator 225. One example of a power source can be a rechargeable battery. As will be described in more detail below, an electrical current applied to the coil assembly 204 in the presence of a magnetic field provided by the magnet assembly 205 will result in a generation of a mechanical force capable of moving the coil assembly 204 and exerting work on the plunger 226 of the syringe 212. The electromagnetic actuator is an efficient force transducer supporting its portability. An exemplary device described in more detail below expends about 50 Joules of energy to deliver about 50-200 micro-liters of a liquid to the eye. For comparison, a standard 9-volt batter can provide up to about 8,500 Joules.

A controller 208 is electrically coupled between the power source 206 and the actuator 225, such that the controller 208 can selectively apply, withdraw and otherwise adjust the electrical input signal provided by the power source 206 to the actuator 225. The controller 208 can be a simple switch that is operable by a local interface or from the housing 140. For example, a button provided on the housing 202 or the housing 140, if the needle-free injector 110 is within a housing 140, may be manipulated by a user, selectively applying and removing an electrical input from the power source 206 to the actuator 225. In some embodiments, the controller 208 includes control elements, such as electrical circuits, that are adapted to selectively apply electrical power from the power source 206 to the actuator 225, the electrical input being shaped by the selected application. Thus, for embodiments in which the power source 206 is a simple battery providing a substantially constant or direct current (D.C.) value, can be shaped by the controller to provide a different or even time varying electrical value. In some embodiments, the controller 208 includes an on-board microprocessor, or alternatively an interconnected processor or personal computer providing multifunction capabilities.

In some embodiments, the needle-free injector 110 includes a remote interface 218. The remote interface 218 can be used to transmit information, such as the status of the needle-free injector 110 or of a substance contained therein to a remote source. Alternatively or in addition, the remote interface 218 is in electrical communication with the controller 208 and can be used to forward inputs received from a remote source to the controller 208 to affect control of the actuator 225. One skilled in the art will understand that the remote interface can be coupled to the housing 140, if such a housing 140 is part of the eye injector 100. Alternatively, or in additional, the controller 2078 can be positioned on any suitable area of the eye injector 100.

The remote interface 218 can include a network interface, such as a local area network interface (e.g., Ethernet). Thus, using a network interface card, the needle-free injector 110 can be remotely accessed by another device or user, using a personal computer also connected to the local area network. Alternatively or in addition, the remote interface 218 may include a wide-area network interface. Thus, the needle-free injector 110 can be remotely accessed by another device or user over a wide-area network, such as the World-Wide Web. In some embodiments, the remote interface 218 includes a modem capable of interfacing with a remote device/user over a public-switched telephone network. In yet other embodiments, the remote interface 218 includes a wireless interface to access a remote device/user wirelessly. The wireless interface 218 may use a standard wireless interface, such as Wi-Fi standards for wireless local area networks (WLAN) based on the IEEE 802.11 specifications; new standards beyond the 802.11 specifications, such as 802.16(WiMAX); and other wireless interfaces that include a set of high-level communication protocols such as ZigBee, designed to use small, low power digital radios based on the IEEE 802.15.4 standard for wireless personal area networks (WPANs).

In some embodiments the controller receives inputs from one or more sensors adapted to sense a respective physical property of the eye. For example, the needle-free injector 110 includes a transducer, such as a position sensor 216B used to indicate location of an object's coordinates (e.g., the coil's position) with respect to a selected reference. Similarly, a displacement may be used to indicate movement from one position to another for a specific distance. Beneficially, the sensed parameter can be used as an indication of the plunger's position as an indication of dose. In some embodiments, a proximity sensor may also be used to indicate a portion of the needle-free injector 110, such as the coil, has reached a critical distance. This may be accomplished by sensing the position of the plunger 226, the force-transfer member 210, or the coil assembly 204 of the electromagnetic actuator 225. For example, the turns of the coil can be counted to determine the coil's position.

Other sensors, such as a force transducer 216A can be used to sense the force applied to the plunger 226 by the actuator 225. As shown, a force transducer 216A can be positioned between the distal end of the coil assembly and the force transfer member 210, the transducer 216A sensing force applied by the actuator 225 onto the force-transfer member 210. As this member 210 is rigid, the force is directly transferred to the plunger 226. The force tends to move the plunger 226 resulting in the generation of a corresponding pressure within the reservoir 213. A positive force pushing the plunger 226 into the reservoir 213 creates a positive pressure tending to force a substance within the reservoir 213 out through the nozzle 214. A negative force pulling the plunger 226 proximally away from the nozzle 214 creates a negative pressure or vacuum tending to suck a substance from outside the device through the nozzle 214 into the reservoir 213. The substance may also be obtained from an ampoule, the negative pressure being used to pre-fill the reservoir 213 with the substance.

An electrical sensor 216C can also be provided to directly sense the pressure applied to a substance within the chamber.

An electrical sensor 216C may also be provided to sense an electrical input provided to the actuator 225. The electrical may sense one or more of coil voltage and coil current. The sensors 216A, 216B, 216C (generally 216) are coupled to the controller 208 providing the controller 208 with the sensed properties. The controller 208 may use one or more of the sensed properties to control application of an electrical input from the power source 206 to the actuator 225, thereby controlling pressure generated within the syringe 212 to produce a desired transfer performance. For example, a position sensor can be used to servo-control the actuator 225 to pre-position the coil assembly 204 at a desired location and to stabilize the coil 104 once positioned, and conclude an actuation cycle. Thus, movement of the coil assembly 204 from a first position to a second position corresponds to transfer of a corresponding volume of substance. The controller can include a processor programmed to calculate the volume based on position give the physical size of the reservoir.

An actuation cycle described in more detail below, generally correspond to initiation of an electrical input to the actuator 225 to induce transfer of a substance and conclusion of the electrical input to halt transfer of the substance. A servo-control capability combined with the dynamically controllable electromagnetic actuator 225 enables adjustment of the pressure during the course of an actuation cycle. One or more of the sensors 216 can be used to further control the actuation cycle during the course of the cycle. Alternatively or in addition, one or more of local and remote interfaces can also be used to further affect control of the actuation cycle.

In some implementations, the controller 208 is coupled with one more other sensors (not shown) that detect respective physical properties of the eye, such as the depth of the eye. This information can be used to servo-control the actuator 225 to tailor the injection pressure, and, therefore, the depth of penetration of drug into the eye. The preferred injection depth is approximately 30 millimeters in order to inject into the retina of the eye 130. The depth however, can be adjusted since different animal's may have slight different dimensions and depths of the eye 130. The injection pressure can be adjusted, for example, by controlling the electrical input signal applied to the actuator 225 and/or the current pulse rise time and/or duration. Moreover, the injection pressure may be varied over time. For instance, in some implementations, a large injection pressure is initially used to pierce the eye with the drug, and then a lower injection pressure is used to deliver the drug. A larger injection may also be used to break a seal that seals the chamber or vial that holds a substance.

In more detail, the power source 206 can be external to the housing 140 of the eye injector 100 if the housing 140 is present. For example, the eye injector 100 can be coupled to a separate electrical power supply. Preferably, however, the power source 206 is self-contained within the eye injector 100 to promote portability of the eye injector 100.

The power source 206 can include a replaceable battery, such as a ubiquitous 9-volt dry cell battery. Alternatively, the power source 206 includes a rechargeable device, such as a rechargeable battery (e.g., gel batteries; lead-acid batteries; Nickel-cadmium batteries; Nickel metal hydride batteries; Lithium ion batteries; and Lithium polymer batteries). In some embodiments, the power source 206 includes a storage capacitor. For example, a bank of capacitors can be charged through another power source, such as an external electrical power source.

In more detail, the electromagnetic actuator 225 includes a conducting coil assembly 204 disposed relative to a magnetic field, such that an electrical current induced within the coil results in the generation of a corresponding mechanical force. The configuration is similar, at least in principle, to that found in a voice coil assembly of a loud speaker. Namely, the relationship between the magnetic field, the electrical current and the resulting force is well defined and generally referred to as the Lorentz force law. Electromagnetic actuators are described in U.S. application Ser. No. 11/352,916, filed Feb. 10, 2006 claiming the benefit of U.S. Provisional Application 60/652,483, filed Feb. 11, 2005, herein incorporated by reference in their entirety.

Preferably, the coil 204 is positioned relative to a magnetic field, such that the magnetic field is directed substantially perpendicular to the direction of one or more turns of the coil 204. Thus, a current induced within the coil 204 in the presence of the magnetic field results in the generation of a proportional force directed perpendicular to both the magnetic field and the coil (a relationship referred to as the “right hand rule”).

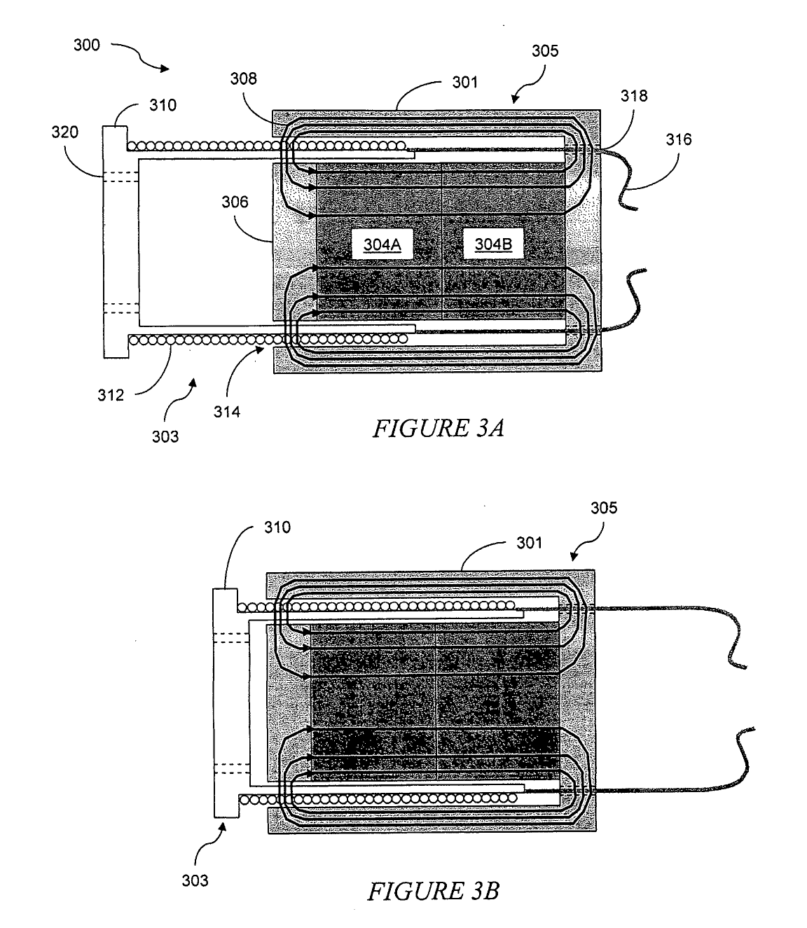

In more detail a cross-sectional diagram of an electromagnetic impulse actuator 225 is shown in FIG. 3A. The device 300 includes a magnet assembly 301 defining an annular slotted cavity 314 and a coil assembly 303 slidably disposed therein. The stroke of the coil 303 can be controlled by the lengths of the coil and magnet assembly. Thus, the electromagnetic actuator can be configured to transfer a substantial volume of a substance during a single, sustained stroke.

Advantageously, the controllability of the actuator also allows for a precise transfer. For example, a substance may be delivered to an eye with minimum volumetric increments of about 1%. Thus, for a 200 micro-liter volume, the dosage may be tailored in 200 nano-liter steps. Thus, a single syringe loaded with a sufficient volume can deliver various doses by controlling the electrical input to the coil. Operation of such an actuator is deterministic further lending itself to precision control.

The magnet assembly 305 includes a column of magnets 304A, 304B disposed along a central axis 303. The column of magnets can be created by stacking one or more magnetic devices. For example, the magnetic devices can be permanent magnets. As a greater magnetic field will produce a greater mechanical force in the same coil, thus stronger magnets are preferred. As portability and ease of manipulation are important features for an eye injector 100 if configured to be hand-held, high-density magnets are preferred.

One such category of magnets are referred to as rare-earth magnets, also know as Neodymium-Iron-Boron magnets (e.g., Nd₂Fe₁₄B). Magnets in this family are very strong in comparison to their mass. Currently available devices are graded in strength from about N24 to about N54—the number after the N representing the magnetic energy product, in megagauss-oersteds (MGOe). In one particular embodiment, N50 magnets are used. The magnetic field produced by the magnets generally follows field lines 308, with rotational symmetry about the central axis for the configuration shown.

The magnets 304A, 304B are attached at one end of a right-circular cylindrical shell 301 defining a hollowed axial cavity and closed at one end. An annular slot remains being formed between the magnets 304A, 304B and the interior walls of the case and accessible from the other end of the shell 301. An exemplary shell 301 is formed with an outside diameter of about 40 mm and an inside diameter of about 31.6 mm, resulting in a wall thickness of about 4.2 mm. In this embodiment, the magnets 304A, 304B are cylindrical, having a diameter of about 25.4 mm.

The shell 301 is preferably formed from a material adapted to promote containment therein of the magnetic fields produced by the magnets 304A, 304B. For example, the shell 301 can be formed from a ferromagnetic material or a ferrite. One such ferromagnetic material includes an alloy referred to as carbon steel (e.g., American Iron and Steel Institute (AISI) 1026 carbon steel). An end cap 306 is also provided of similar ferromagnetic material being attached to the other end of the magnets 304A, 304B. Placement of the end cap 306 acts to contain the magnetic field therein and promoting a radially-directed magnetic field between the annular gap formed between the end cap 306 and the outer walls of the shell 301. The end cap is generally thicker than the shell walls to promote containment of the magnetic fields as they loop into the end of the top magnet 304A. For the exemplary shell 301 embodiment described above, the end cap 306 has an axial thickness of about 8 mm.

The coil assembly 303 includes a coil 312 formed from a conducting material, such as copper wire wound about a bobbin 310. The bobbin 310 can be cylindrical and defines an axial cavity sized to fit together with the coil 312 within the annular cavity 314. In some embodiments, the bobbin 310 is substantially closed at the end juxtaposed to the annular cavity 314. The closed end forms a force-bearing surface adapted to push against a plunger 214 (FIG. 1) or force-bearing rod 210 (FIG. 2).

Preferably, the bobbin 310 is formed from a strong, yet light-weight material such as aluminum or epoxy-loaded fiberglass. One such family of glass reinforced epoxy is sold under the trade name GAROLITE®. Suitable material selected from this family includes G10/FR4 material offering extremely high mechanical strength, good dielectric loss properties, and good electric strength properties, both wet and dry. Other materials include an all-polymeric reinforced, dull gold colored polytetrafluoroethylene (PTFE) compound that operates exceptionally well against soft mating surfaces such as 316 stainless steel, aluminum, mild steel, brass and other plastics available from Professional Plastics of Fullerton Calif. under the trade name RULON®. The bobbin 310 is thin-walled to fit within the annular slot. The bobbin 310 should also present a low coefficient of friction to those surfaces that may come in contact with either the shell 301, the magnets 304A, 304B or the end cap 306. In some embodiments, a low-friction coating can be applied to the bobbin. Such coatings include fluorocarbons, such as PTFE.

Generally, a non-conducting material such as epoxy-loaded fiberglass is preferred over a conducting material such as aluminum. Eddy currents created in the conducting material as it moves through the magnetic field tend to create a mechanical force opposing motion of the bobbin. Such an opposing force would counteract intentional movement of the coil thereby resulting in an inefficiency. Dielectric materials reduce or eliminate the production of such eddy currents.

A thin-walled bobbin 310 allows for a narrower annular slot 314 thereby promoting a greater magnetic field intensity across the gap. A substantial current flowing within the coil 312 can result in the generation of a substantial thermal load that could result in structural damage (e.g., melting). Other light-weight materials include machinable poly-acetals, which are particularly well suited to high-temperature applications.

Continuing with the exemplary embodiment, the bobbin 310 has an outside diameter of about 27 mm, an internal diameter of about 26 mm, and an axial length of about 46 mm. The coil 312 consists of six layers of 28 gauge copper wire wound onto the bobbin 310 at a rate of about 115 windings per coil length (about 35 mm) resulting in about 700 turns total. Using the N50 magnets with the 1026 carbon steel, the end cap 206 contains between about 0.63 and 0.55 Tesla (the value reducing outwardly along a radius measured from the center of the end cap 206).

Thus, a current flowing through the coil 312 is positioned at right angles to the magnetic field 208 produced between the end cap 206 and the shell 201 wall. This results in the generation of a force on the coil directed along the longitudinal axis, the direction of the force depending upon the directional flow of the current. For the above exemplary device, an electrical input, or drive voltage of about 100 volts applied across the coil for a duration of about 1 millisecond representing the pierce phase of an actuation cycle. A lesser electrical input of about −2 volts is applied for the transfer phase.

Generally, the coil 312 receives the electrical input signal through two electrical leads 316. The shell 301 includes one or more apertures 318 through which the leads 316 are routed to the power source 206 (FIG. 2). The closed end of the shell 201 may contain one or more additional apertures through which air may be transferred during movement of the coil. Without such apertures and given the relative tight tolerances of the gap between the coil 312 and the annular slot 314, a pressure would build up to oppose movement of the coil. Alternatively or in addition, the bobbin 310 may also have one or more apertures 320 to further inhibit the build up of damping pressures during actuation.

FIG. 3A shows the coil assembly 303 after or during an injection phase in which the coil is forced out of the shell 301 thereby advancing the front plate 315. FIG. 3B shows the coil assembly 303 retracted within the shell 301 after a sampling phase in which the coil assembly 303 is drawn into the shell 301.

In some embodiments, the conductive coil is configured to carry a relatively high-amplitude electrical current to produce a substantial force resulting in the generation of a substantial pressure. The coil also provides a relatively low inductance to support high-frequency operation thereby enabling rapid rise time (i.e., impulse) response. In some embodiments, the coil provides an inductance of less than 100 millihenries. Preferably, the coil inductance is less than about 50 millihenries. More preferably, the coil inductance is less than about 10 millihenries. For example, the coil inductance can be between about 4 and 10 millihenries. One way of providing the high-current capacity with the low inductance is using a coil formed by a large-diameter conductor that is configured with a low number of turns (e.g., 1 to 3 turns).

The result is an eye injector 100 with a pressure actuator capable of generating a high-pressure pulse with a rapid rise time. Additionally, operation of the actuator is both controllable and highly predictable given the physical properties of the actuator and the input electrical current. Still further, the actuator is reversible providing forces in opposing directions based on the direction of current flow within the coil.

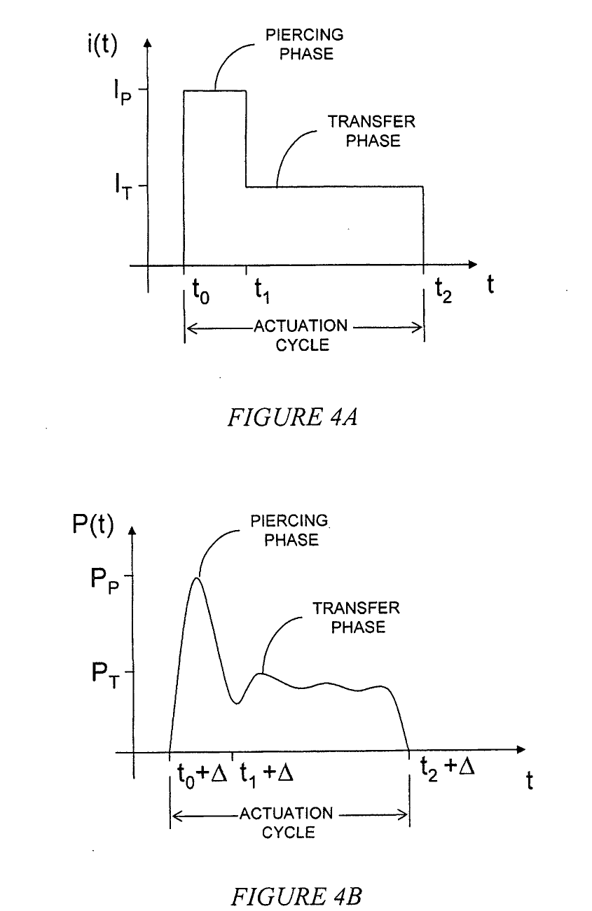

Additionally, the controllability allows for a tailored injection profile that can include a rapid high-pressure pulse to breach the eye, followed by a lower-pressure, prolonged pulse to deliver the formulation to the eye. Referring to FIG. 4A, an exemplary time varying electrical input is shown. The curve represents variation in an electrical current applied to the coil assembly 204 of the actuator 225. At a first instant of time to an electrical current is applied to the coil 204. The current rises from a rest value (e.g., zero amps) to a maximum value I_{P} remaining at this maximum for a selectable duration and then transitioning to a different current value I_{T} at a later time t₁. The current amplitude may remain substantially at this value, or continue to vary with time until a later time t₂, at which the current returns to a rest value.

The entire period of time defined between times t₂ and t₀ can be referred to as an actuation period, or actuation cycle. For a current input having a shape similar to that just described, the period defined between times t₁ and t₀ can be referred to as a piercing phase. As the name suggests, the high current value I_{P} induces a corresponding high pressure that can be used to pierce the surface of the eye without using a needle or lance. The remainder of the actuation cycle defined between times t₂ and t₁ can be referred to as a transfer phase. As this name suggests, the relatively lower current value I_{T} induces a lesser pressure that can be used to transfer a substance from the reservoir 213 (FIG. 2) to the biological body through the perforation in the surface created during the piercing phase.

An exemplary plot of a pressure induced within the reservoir 213 (FIG. 2) in response to the electrical input is illustrated in FIG. 4B. As shown, the pressure rises from an initial rest value to a relative maximum value, P_{P}, at a time t₀, perhaps with a slight delay Δ resulting from the transfer characteristics of the electrical coil. This pressure value can be used to create a jet as described above in relation to FIG. 1. As the current is reduced during the transfer phase, the pressure similarly reduces to a lesser value P_{T} determined to achieve a desired transfer of the substance. The transfer phase continues until a desired volume of the substance is transferred, then the pressure is removed concluding the actuation cycle.

A servo-controlled injector includes a specially-designed electromagnetic pressure actuator configured in combination with a servo controller to generate an injection pressure responsive in real-time to one or more physical properties (e.g., pressure, position, volume, etc.). In some embodiments, the servo-controlled injector is a needle-free device. The electromagnetic pressure actuator generates a high-pressure pulse having a rapid rise time (e.g., less than 1 millisecond) for injecting a formulation into the eye. The pressure provided by the actuator can be varied during the actuation of a single injection to achieve a desired result. For example, a first high-pressure is initially provided to the formulation to penetrate the outer surface layer of an eye. Once the eye is penetrated, the pressure is reduced to a second, lower pressure for the remainder of the injection. The servo-controller can be used to determine when the skin is penetrated by sensing a change in pressure within the chamber and to adjust the injection pressure accordingly.

A servo-controller 208 receives input signals from the one or more sensors 216 and generates an output signal according to a predetermined relationship. The servo-controller output can be used to control the pressure by controlling the amplitude of electrical current driving the controllable actuator.

Real-time control can be accomplished by the servo controller 208 repeatedly receiving inputs from the sensors 216, processing the inputs according to the predetermined relationship and generating corresponding outputs. In order to adjust the injection pressure during the course of an injection, the entire sense-control process must be performed numerous times during the period of the injection. For example, a servo-controller 208 can include a high-speed microprocessor capable of processing signals received from the sensors and rapidly providing corresponding output signals at a rate of 100 kHz (i.e., every 10 microseconds). Such rapid response times provide hundreds of opportunities to adjust pressure during the course of a single 5 to 10 millisecond injection.

As friction or drag on the coil assembly 204 represents an inefficiency, the coil can be floating within a cavity of the magnet assembly 205. That is, there is the coil assembly 204 floats within a gap and is allowed to move freely. With no current applied to the coil assembly 204, it would be allowed to slide back and forth with movement of the needle free injector 110 of the eye injector 100. Such movement may be undesirable as it may result in unintentional spillage of a substance form the reservoir or introduction of a substance, such as air, into the reservoir. Using a servo-controller with the position sensor 216B, the position of the coil 204 can be adjusted such that the coil 204 is held in place in the presence of external forces (e.g., gravity) by the application of equal and opposite forces induced from the electrical input signal applied to the coil assembly 204.

Alternatively or in addition, the actuator 225 can be coupled to a bellows forming a chamber containing a formulation. For either configuration, actuation results in the generation of a pressure within the chamber, the chamber forcing the formulation through a nozzle.

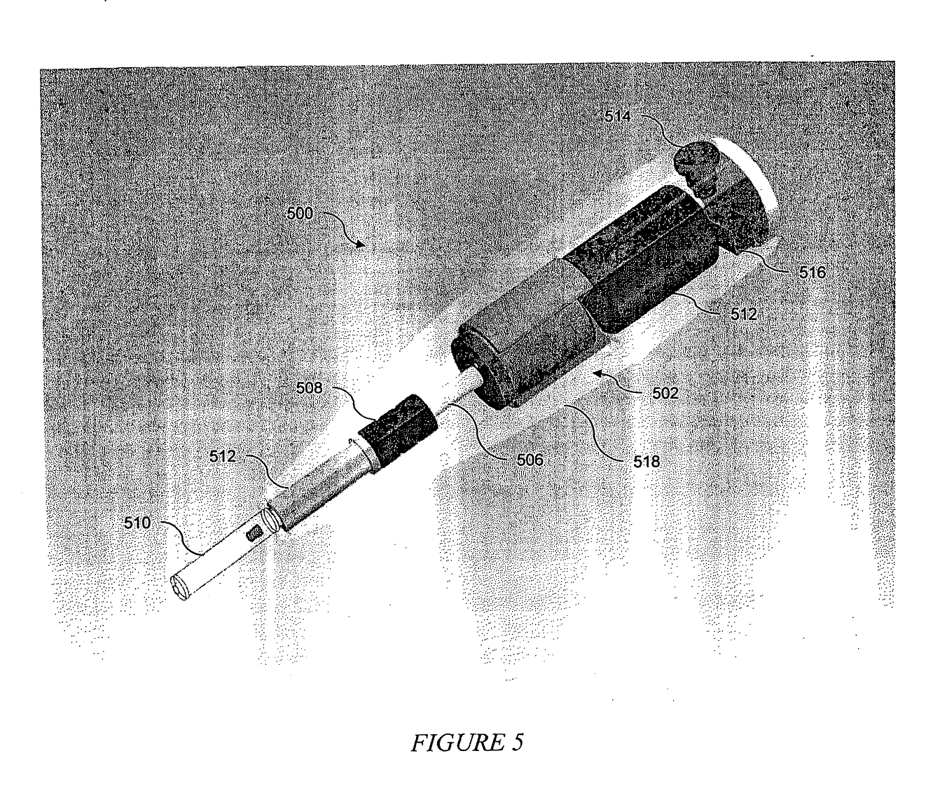

An exemplary embodiment 500 of the needle-free injector 110 of the eye injector 100 is shown in FIG. 5. The needle-free injector 110 includes a controllable electromagnetic actuator 502 abutting one end to a pusher rod 506. The axis of the pusher rod 506 is collinear with the longitudinal axis of the actuator 502 and slides through a bearing 508 to inhibit radial movement. A mounting adapter 512 is provided at a distal end of the device 500 for mounting a syringe 510. A plunger of the syringe (not shown) resides within the mounting adapter 512 abutting the other end of the pusher rod 508. A power source, such as a rechargeable capacitor 512 is disposed proximal to the actuator 502 for inducing currents within the actuator 502. The needle free injector 500 also includes a button 514 to initiate an injection and a controller 516 to control application of the power source to the actuator 502. The button 514 can alternatively be positioned on the housing 140 of the eye injector, or on any suitable position on the eye injector 100. A housing, such as an elongated molded plastic case 518 is also provided to secure the different components with respect to each other.

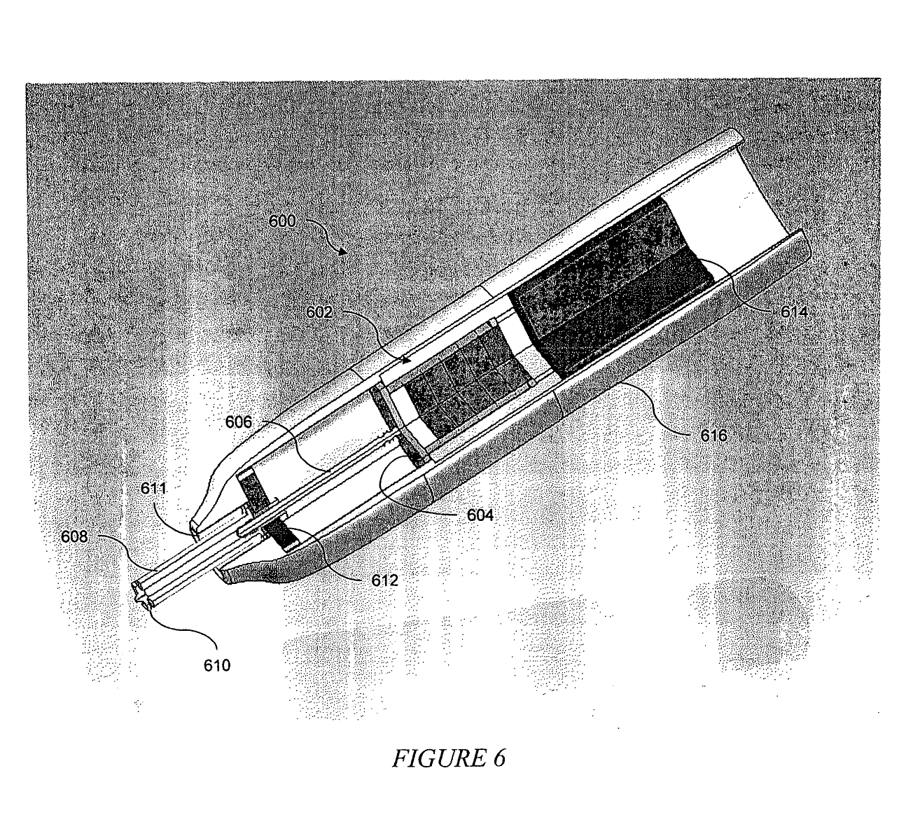

An exemplary embodiment 600 of a smaller, needle free injector 110 of the eye injector 100 is shown in FIG. 6. The device 600 includes a compact electromagnetic actuator 602 having a distal force plate 604 adapted to abut a proximal end of a plunger 606 of a syringe 608. The needle-free injector 600 also includes a mounting member 612 to which a proximal end of the syringe 608 is coupled. A power source 614 is also disposed proximal to the actuator 602, the different components being secured with respect to each other within a housing 616.

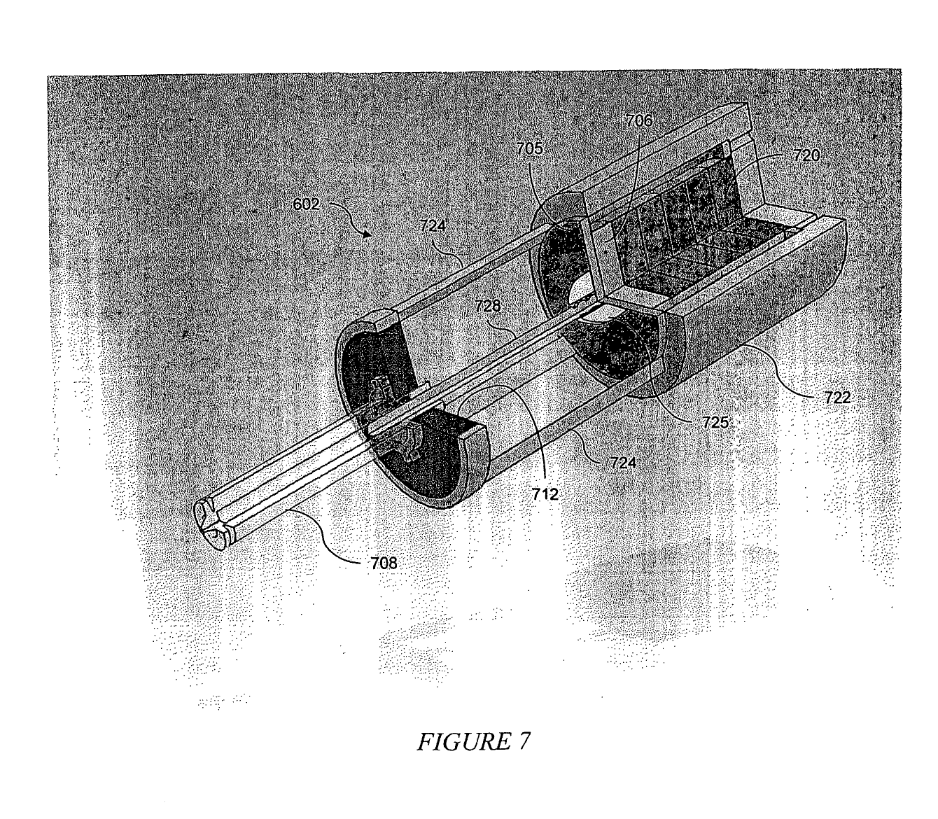

Referring to FIG. 7, in more detail, the compact controllable electromagnetic actuator 602 includes a ferromagnetic shell 722 including a central magnetic core 720 capped by a ferromagnetic end cap 706. A coil assembly 705 is slidably disposed within an annular slot of the magnet assembly floating freely within the slot. The distal end of the shell 722 includes one or more extensions 724 that continue proximally from the distal end of the shell 722 and terminating at the distal mounting plate 712. The interior surface of these extensions 724 provides a bearing for the coil assembly 705 allowing axial movement while inhibiting radial movement. The extensions 724 may include openings between adjacent extensions 724 as shown to reduce weight and to promote the flow of air to promote coil movement and for cooling. This configuration rigidly couples the distal mounting plate 712 to the shell 722, thereby increasing rigidity of the actuator 602 and reducing if not substantially eliminating any stress/strain loading on the housing 618 (FIG. 6) caused by actuation of the device.

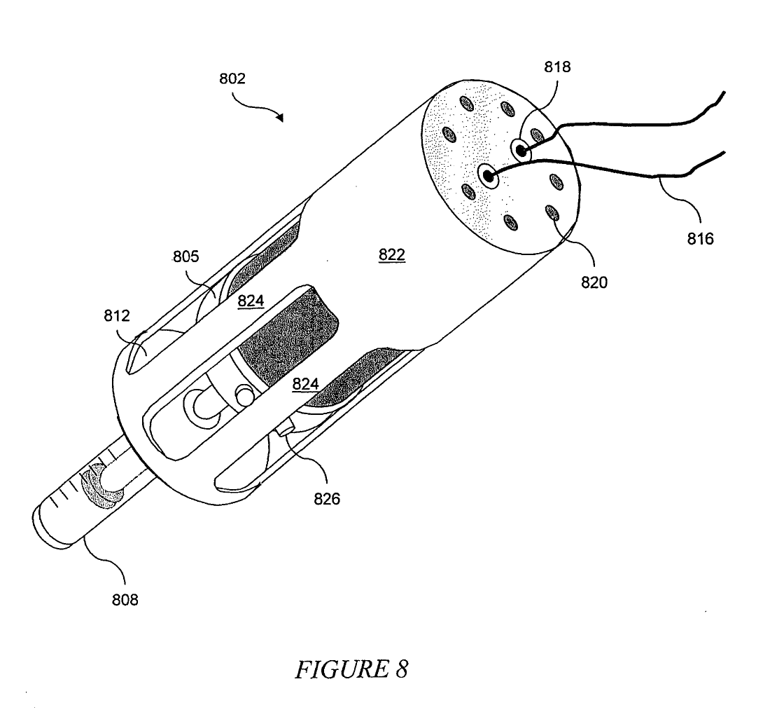

A rear perspective view of an exemplary compact Lorentz-force actuator 802 is shown in FIG. 8. The needle free injector 802 includes a magnet assembly having an external shell 822. A coil assembly 805 is slidably disposed within the shell 822, and adapted for axial translation. Multiple longitudinal extensions 824 are disposed about the axis and adapted to couple the shell 822 a mounting plate 812. Openings are provided between adjacent extensions 824. A syringe 808 is coupled to the mounting plate 812 at the distal end of the device 802. One or more guides 826 are provided to prevent rotation of the coil, each guide 826 riding along an interior edge of an adjacent extension 824. The proximal end of the device 802 includes apertures 818 through which the coil leads 816 are routed and one or more additional apertures 820 to promote air flow during actuation

Because the Lorentz-force actuator is bi-directional, depending upon the direction of the coil current, the same device used to inject a substance can also be used to withdraw a sample. This is a beneficial feature as it enables the device to collect a sample. For example, the eye injector 100 could be used to withdraw material from the aqueous humor of the eye for genetic, protein and fluid analyte analysis.

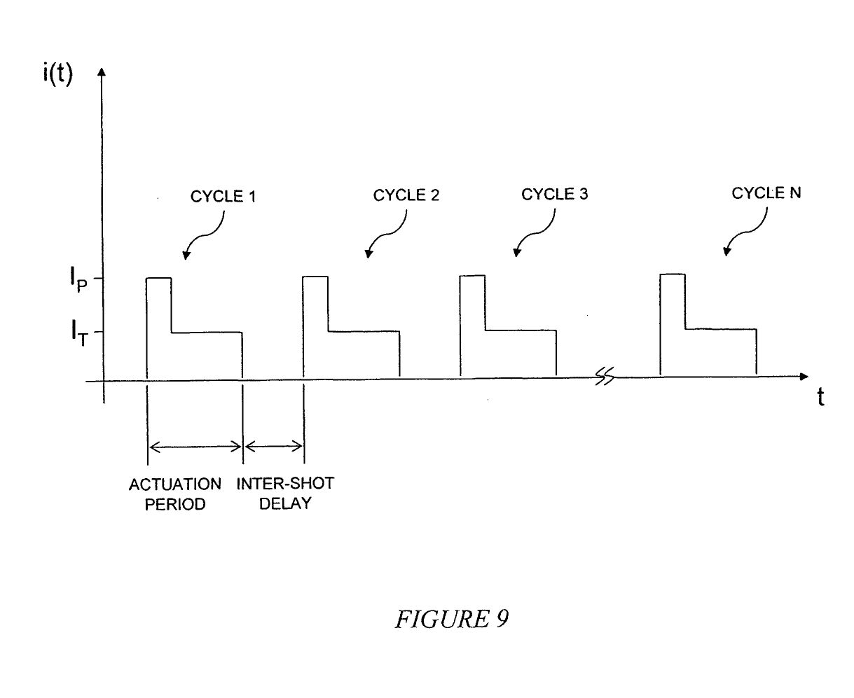

In some embodiments, it is advantageous to provide a controllable needle-free eye injector capable of administering multiple injections and/or samples in succession. Thus, actuation cycles occur with relatively short time delay between cycles adjacent. Such a device can be referred to as a multi-shot needle-free eye injector. Multi-shot injections can occur within 30 milliseconds to 50 milliseconds per cycle, with an actuation (i.e., injection) cycle 10 milliseconds. Some multi-shot devices have a capability to deliver up to 500 injections per drug vial. A plot of an exemplary coil drive current versus time for a multi-shot application is illustrated in FIG. 9.

Further, the operational features offered by the dynamically controllable Lorentz-force actuator support numerous and varied treatment options. Combining both a forceful injection capability with controllability, the same controllable eye injector 100 can be used to deliver varied injections. For example, the device can be used non-invasively to deliver into different depths of in the eye to transfer a substance.

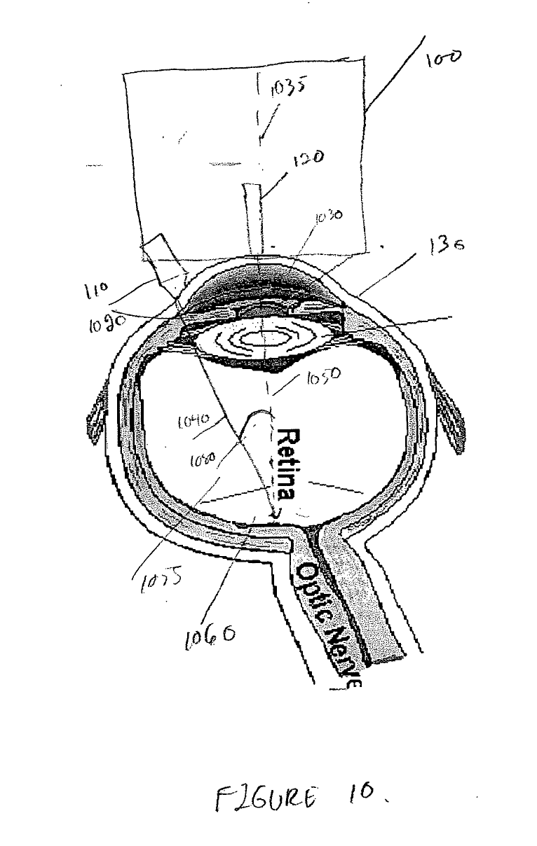

The operation of the eye injector 100 is shown in FIG. 10. The eye injector 100 is placed on the surface of the eye 130. The needle-free injector 110 is positioned adjacent to the lens 1020 of the eye 130. The targeting light source 120 is positioned parallel to the major axis 1035 of the pupil 1030. The needle-free injector creates a vector 1040. The targeting light beam creates a straight line 1050 that intersects with the vector 1040 to create an angle 1080. A substance is then injected as a jet along the intersecting path of the light beam 130 and the vector 1040. The substance can be injected along the intersecting vector 1040, or alternatively can be injected within the angle 1080. Injecting along the intersecting vector 1040 or within the angle allows the substance to be delivered to the retina 1060. The substance is carried through the vitreous humor 1055 to the retina 1060 of the eye 130. Advantageously, because of the positioning of the injector 110 adjacent to the lens and not on the lens, the lens 1020, which is a sensitive area of the eye 130, is not pierced if injection is along the intersecting vector 1040.

Alternatively, if penetration of the lens 1020 is desired, the needle-free injector e 110 may be positioned on the lens 1020. Additionally, the position of the targeting light source 1020 and the needle-free injector 110 may be reversed, wherein the targeting light source 120 is placed adjacent to the lens 1020 while the needle-free injector is placed on or near the pupil of the eye. Regardless of the positioning, the targeting light beam 120 and the vector produced by the needle-free injector should intersect an angle 1080. The substance should be delivered along this angle 1080 in order to reach the retina.

Further, the needle-free injector 110 does not have to be positioned at an angle on the eye. The needle-free injector 110 can be positioned parallel to the major axis of the eye 130. The orifice of the injector, however, is then placed laterally on the nozzle, thus still producing a vector 1040 that intersects with the targeting light beam 130, thus producing the angle 1080. The jet stream of the substance is then injected along the angle 1080 as described.

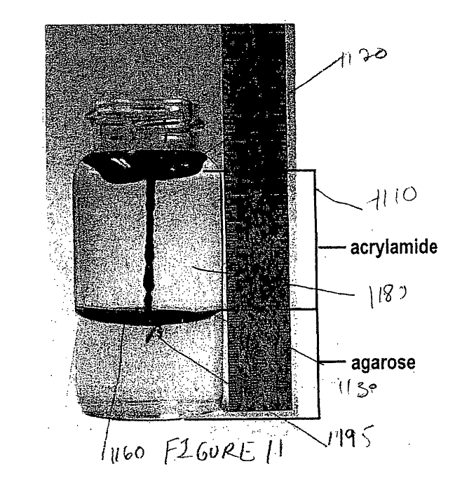

A simulated model of the process is shown in FIG. 11. Acrylamide 1110 is used to simulate the lens 1120 and the retina of the eye, while agarose 1130 is used to simulate the vitreous humor 1140 of the eye. A dye 1140 represents the substance being injected. The dye 1140 when injected forms a jet and travels towards the retina, where it spreads at 1195.

The depth of the normal eye of an animal between the lens and the retina is approximately 30 millimeters as can be seen. The jet is therefore, carried approximately 30 mm into the eye 130. The depth can be adjusted based on the depth of individual eyes by the user, by manipulating a controller of the eye injector 100 as previously described.

The eye injector 100 can be coupled a patient positioning mechanism. The patient positioning mechanism allows the eye injector 100 to be properly placed on the eye and immobilized for the purpose of the injection. The eye injector 100 may be coupled to various optical instruments for use independently or in conjunction with different optical procedures.

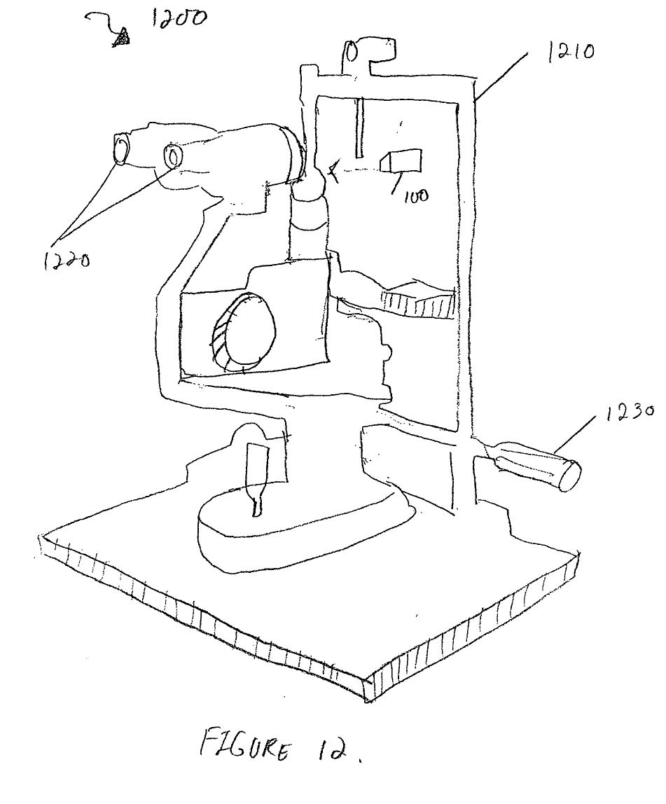

For example, the eye injector 100 can be coupled to a slit lamp photometer 1200, as shown in FIG. 12. The slit lamp photometer 1200 includes a patient positioning mechanism 1210, which can assist in adjusting the binoculars 1220 against the head of the patient. The patient positioning mechanism 1210 can include a frame with two upright metal rods to which are attached a forehead strap and a chin rest. A lens of the binocular may be removed and the eye injector 100 may be placed, therein, while the patient positioning mechanism 1210 is maneuvered by the user such that the eye injector rests against the surface of the eye as needed. A lever 1230 can be used to adjust the height of the binoculars as well. Also, if an injection is needed in both eyes, two eye injectors 100 can be placed in both lenses of the binoculars 1220. The eye injector, may for example, be used in conjunction with the ZEISS® slit lamp or additional optical instruments produced by ZEISS®. In one embodiment, the optical instrument can provide the targeting beam of light and the beam of the light 120 of the eye injector need not be used.

The eye injector 100 can be useful in treating several diseases of the eye. For example, Age Related Macular Degeneration is one of the leading causes of blindness in the United Stated. A drug, Macugen® is currently used to slow vision loss. Macugen® is injected into the macular region of a patient's eye with a needle every six weeks. This injection is normally made with a 27 guage or approximately 4 millimeter diameter needle. Complications of this type of injections include endophthalmitis, retinak detachment, hemorrhage in the eye, iatrogenic damage to other ocular structures in the eye such as the lens, and various types of infections at the site of the injection.

The needle-free eye-injector 100 can be used as a safer means for delivering Macugen® to the macular region of a patient's eye. A needle-free injection produces a much smaller hole in the eye (from about 100 μm-200 μm) than an injector using a needle (about 400 μm), thus greatly reducing the chances of infection, or potential damage to other areas of the eye with the needle. Additionally, a needle-free injection also limits a patient's anxiety of having a needle injected into his or her eye.

The eye injector 100 can also be used for treating various diseases of the eye or the brain, such as Alzheimer's' disease. A Nerve growth factor (NGF) a type of neurotrophic found in the periphery area. NGF does not significantly penetrate the blood-brain barrier, which makes its clinical utility dependent on invasive neurosurgical procedures. When conjugated to an antibody, however, NGF can cross the blood-brain barrier after peripheral injection. The eye injector 100 would allow for a non-invasive transfer of antibodies, which could attach to the NGF and move across the blood-brain barrier, thus being carried to the brain with reduced chances of damage and infection of the eye. This approach may prove useful for the treatment of Alzheimer's disease and other neurological disorders that are amenable to treatment by proteins that do not readily cross the blood-brain barrier.

The controllable nature of such an eye injector lends itself to automatic, or robotic injection. As the injection is needle-free, there is no chance of a needle breaking within the eye of animal, should the animal move during the course of an injection. Further, because a forceful needle-free injection can be accomplished in a fraction of a second, the duration of time during which an animal must remain immobile is greatly reduced.

While this invention has been particularly shown and described with references to preferred embodiments thereof, it will be understood by those skilled in the art that various changes in form and details may be made therein without departing from the scope of the invention encompassed by the appended claims.

What is claimed is:

1. A delivery system for transferring a substance into an eye of an animal comprising;
a needle-free injector comprising;
a reservoir for storing the substance;
a nozzle in fluid communication with the reservoir; and
an actuator in communication with the reservoir, the actuator driving the substance from the reservoir to the nozzle; and
a patient positioning mechanism to position the needle-free injector relative to an eye of a patient to direct the needle-free injector to inject a jet into the eye.

2. The delivery system of claim 1, further comprising a targeting light source that directs a beam of light through the pupil of the eye.

3. The delivery system of claim 1, wherein the needle-free injector is positioned adjacent to a lens of the eye.

4. The delivery system of claim 1, wherein the needle-free injector allows the depth of injection to be controlled.

5. The delivery system of claim 1, wherein the substance is injected to depth of about 30 millimeters into the eye.

6. The delivery system of claim 1, wherein the substance is a drug.

7. The delivery system of claim 1, wherein the substance is a powder.

8. The delivery system of claim 1, wherein the delivery of a substance into the eye can be made in between 30-50 milliseconds.

9. The delivery system of claim 1, wherein the actuator is a controllable electromagnetic actuator.

10. The delivery system of claim 2, wherein the beam of light and the jet of the needle-free injector intersect at a point at or near a retina of the eye.

11. The delivery system of claim 2, wherein the source of light is a laser.

12. The delivery system of claim 9, wherein the electromagnetic actuator further comprises:
a stationary magnet assembly providing a magnetic field; and
a coil assembly, slidably disposed with respect to the magnet assembly, the coil assembly receiving an electrical input and generating in response a force corresponding to the received input, the force resulting from interaction of an electrical current within the coil assembly and the magnetic field and causing a needle-free transfer of the substance between the reservoir and the eye.

13. The delivery system of claim 12, wherein the electromagnetic actuator forces the substance through the nozzle producing the jet having sufficient velocity to pierce the surface of the eye.

14. The delivery system of claim 12, wherein the electromagnetic actuator is a Lorentz force actuator.

15. The delivery system of claim 12, wherein a rise-time of the generated force is less than about 5 milliseconds.

16. The delivery system of claim 12, wherein the force is of sufficient magnitude and duration to transfer a volume of up to at least about 300 micro liters of the substance.

17. A device for transferring a substance into an eye of an animal comprising;
a needle-free injector comprising;
a reservoir for storing the substance;
a nozzle in fluid communication with the reservoir; and
an actuator in communication with the reservoir, the actuator driving the substance from the reservoir to the nozzle; and
a light source to emit a targeting beam of light into the eye, the beam of light being coupled to the needle free injector.

18. The device of claim 17, wherein the needle-free injector is an accessory to a patient positioning mechanism to position the needle-free injector against an eye of a patient to direct the needle-free injector to inject a jet into the eye.

19. The device of claim 17, wherein the needle-free injector is positioned adjacent to a lens of the eye.

20. The device of claim 17, wherein the beam of light and a jet of the needle-free injector intersect at a point at or near a retina of the eye.

21. The device of claim 17, wherein a depth of injection is controllable.

22. The device of claim 17, wherein the substance is injected to depth of about 30 millimeters into the eye.

23. The device of claim 17, wherein the source of light is a laser.

24. The device of claim 17, wherein the substance is a drug.

25. The device of claim 17, wherein the substance is a powder.

26. The device of claim 17, wherein the actuator is a controllable electromagnetic actuator.

27. The device of claim 17, wherein the injection of a substance into the eye can be made between 30-50 milliseconds.

28. The device of claim 18, wherein the patient positioning mechanism is a slit lamp.

29. The device of claim 26, wherein the electromagnetic actuator further comprises:
a stationary magnet assembly providing a magnetic field; and
a coil assembly, slidably disposed with respect to the magnet assembly, the coil assembly receiving an electrical input and generating in response a force corresponding to the received input, the force resulting from interaction of an electrical current within the coil assembly and the magnetic field and causing a needle-free transfer of the substance between the reservoir and the eye.

30. The device of claim 29, wherein the electromagnetic actuator forces the substance through the nozzle producing a jet having sufficient velocity to pierce the surface of the eye.

31. The device of claim 29, wherein a rise-time of the generated force is less than about 5 milliseconds.

32. The device of claim 29, wherein the force is of sufficient magnitude and duration to transfer a volume of up to at least about 300 micro liters of the substance.

33. A needle free injection device for transferring a substance into an eye of an animal comprising;
a reservoir for storing the substance;
a nozzle in fluid communication with the reservoir, the nozzle positioned adjacent to a lens of the eye;
an actuator in communication with the reservoir, the actuator driving the substance from the reservoir to the nozzle; the actuator causing a needle-free transfer of the substance between the nozzle and the eye to a depth of about 30 millimeters into the eye; and
a targeting light source adapted to emit a beam of light in a straight line into a pupil of the eye.

34. The device of claim 33, further comprising a patient positioning mechanism to position the nozzle against an eye of a patient to direct the nozzle to inject a jet into the eye.

35. The device of claim 33, wherein the actuator is a controllable electromagnetic actuator.

36. The device of claim 33, wherein the nozzle is positioned adjacent to a lens of the eye.

37. The device of claim 33, wherein the beam of light and a jet produced by the nozzle intersect at a point at or near a retina of the eye.

38. The device of claim 34, wherein the patient positioning mechanism is a slit lamp.

39. The device of claim 35, wherein the electromagnetic actuator further comprises:
a stationary magnet assembly providing a magnetic field; and
a coil assembly, slidably disposed with respect to the magnet assembly, the coil assembly receiving an electrical input and generating in response a force corresponding to the received input, the force resulting from interaction of an electrical current within the coil assembly and the magnetic field and causing a needle-free transfer of the substance between the reservoir and the eye.

40. A method of transferring a substance into the eye comprising;
positioning a needle-free injection device at or near a surface of the eye; and
injecting a jet through a vitreous humor of the eye to a retina of the eye.

41. The method of claim 40, wherein the needle-free injector is an accessory to a patient positioning mechanism to position the needle-free injector against an eye of a patient to direct the needle-free injector to inject a jet into the eye.

42. The method of claim 40, wherein the needle-free injector further comprises a controllable electromagnetic actuator.

43. The method of claim 40, wherein the needle-free injector is positioned adjacent to a lens of the eye.

44. The method of claim 40, wherein the needle free injector further comprises a targeting source of light, the targeting source of light emitting a beam of light into a pupil of a patient's eye.

45. The method of claim 40, wherein the beam of light and a jet produced by the needle-free injector form an angle along which a substance is injected into the eye.

46. The method of claim 40 wherein the injection can be used for treating Alzheimer's disease.

47. The method of claim 42, wherein the electromagnetic actuator further comprises:
a stationary magnet assembly providing a magnetic field; and
a coil assembly, slidably disposed with respect to the magnet assembly, the coil assembly receiving an electrical input and generating in response a force corresponding to the received input, the force resulting from interaction of an electrical current within the coil assembly and the magnetic field and causing a needle-free transfer of the substance between the needle-free injector and the eye.

48. A method of transferring a substance into an eye of an animal comprising;
causing a light source to emit a targeting beam of light into a pupil of the eye;
positioning a needle-free injector adjacent to a lens of the eye;
applying an electrical input to a Lorentz force actuator, the Lorentz force actuator receiving the input and generating in response a force proportional to the received input, the force being capable of causing a needle-free injection of the substance; and
injecting the substance from the needle-free injector along or within an angle, the angle created by the intersection of a jet produced by the needle-free injector and the targeting beam of light, the substance being injected approximately 30 millimeters into the eye at or near the retina of the eye.

49. A method of moving a substance between the eye of an animal and an injection device comprising;
positioning a needle-free transport device at or near a surface of the eye; and
moving the substance through the surface of the eye with the needle-free transport device.

50. A device for transferring a substance into the eye comprising;
a means for positioning a needle-free injection device at or near a surface of the eye; and
a means for injecting a jet through a vitreous humor of the eye to a retina of the eye.
